(19) United States
(12) Patent Application Publication
(10) Pub. No.: US 2002/0138880 A1
(43) Pub. Date: Sep. 26, 2002

(54) ALUMINIUM RESISTANCE GENE

(76) Inventors: RICHARD CLAGUE GARDNER, AUCKLAND (NZ); COLIN WHITI MACDIARMID, AUCKLAND (NZ); ROBERT JOHN HAY, PALMERSTON NORTH (NZ)

Correspondence Address: JACOBSON PRICE HOLMAN & STERN, 400 7TH STREET NORTHWEST, SUITE 600, WASHINGTON, DC, 20004

(21) Appl. No.: 08/945,749

(22) PCT Filed: May 1, 1996
(86) PCT No.: PCT/NZ96/00035

(30) Foreign Application Priority Data
May 1, 1995 (NZ) 272039

(51) Int. Cl. C07H 21/02; C07H 21/04; A61K 31/70; A01N 43/04; C12P 21/06; C12N 1/14; C12N 1/16; C12N 1/18; A01H 9/00; A01H 11/00; C12N 15/00; C12N 15/09; C12N 15/63; C12N 15/70; C12N 15/74; C12N 15/85; C12N 15/87; C12N 15/82

(52) U.S. Cl. 800/295; 435/320.1; 536/23.1; 435/254.2; 514/44; 435/69.1; 435/455; 435/483; 435/468

(57) ABSTRACT

A method of isolating genes conferring resistance to aluminium is provided and two particular aluminium tolerant genes are described. These genes are designated ALR1 and ALR2. The two tolerance genes were isolated from yeast strains but were found to have homology with bacterial genes responsible for divalent ion uptake. Hence a method of isolating divalent cation transporters is envisaged by using complementation of magnesium transporter mnutant strains of yeasts aluminium tolerance genes.

### FIELD OF INVENTION

The invention relates to an aluminium resistance gene, specifically the aluminium resistance gene from S. cerevisiae. More specifically the invention relates to -he isolation and DNA and amino acid sequence of an aluminium resistance gene.

### BACKGROUND OF INVENTION

The aluminium ion has no known biological function, but A1 toxicity is a well documented phenomenon (Kochian 1995). The level of toxic A1 spoecies in solution is largely determined by the pH. At a DH above 5.5, A1 is mostly present as non-toxic aluminium hydroxide or aluminium sulphate complexes. H4owever, in more acid conditions, A1³+becomes the predominant A1 ion in solution, and is generally believed to be the toxic species (Kochain 1995). Plants which are grown in acid soil conditions have reduced root systems, and exhibit a variety of nutrient deficiency symptoms, with consecruent decrease in yield (Luttge et al. 1992). in many developing countries, large land areas are covered by acid soils, making cultivation of many crop plants uneconomic.

Although most of the work on Al toxicity has been in plant systems, Al is also toxic to microorganisms such as bacteria and algae (Date et al. 1970, Pettersson et al. 1989), although less is known about the toxic species involved. Aluminium is also toxic.to fish at low concentrations, causing damage to gill tissues (Baker et al. 1982). In man, aluminium has also been associated with several pathological states, including neurological disorders such as Azheimers disease, and syndromes related to long-term dialysis (MacDonald et al. 1987). The toxicity of A1 to living systems therefore seems to be a general phenomenon.

In plants, the interaction of Al with the cell is modulated by the concentration of other cations in solution (most noticeably Ca and Mg) Low concentrations of these divalent ions are often associated with high levels of A1 in the soil solution (Dahlgren 1994), and increasing the levels of these cations in solution can ameliorate A1 toxicity (Alva et al. 1986, Kinraide et al. 1987). Al toxicity in some plants is associated with lowered uptake of these cations (Rengel e; al. 1988, Rengel 1988) and deficiency symptoms (Foy 1983). Some workers have suggested that A1 is acting to directly inhibit membrane transporter proteins responsible for the uptake of cations such as Ca, Mg and K (Rengel and Robinson 1988, Rengel and Elliott 1992, Gassmann and Schroeder 1994). Low activities of these cations in soil solutions would then be exected to exacerbate A1 toxicity. In addition to cations, other substances (notably organic anions) are known to ameliorate A1 toxicity, probably by chelating free A1 and removing it from solution. Among the most effective ameliorative anions are citrate, malate and EDTA (Suhayda et al. 1986, Conner et al. 1985).

Although A1 has been reported to interact strongly with a number of organic molecules including proteins, polynucleotides and glycosides (MacDonald et al. 1938), (Martin 1992), little progress has been made in elucidation of a definitive mechanism for the inhibitory action of this ion in biological systems. Some workers have proposed that A1 is due to substitution or the A1 ion for divalent cations at the catalytic sites of crucial cellular enzymes or signal transduction proteins (MacDonald et al. 1987, F-aug et al. 1994). One such cellular component which has atracted much attention as a possible target for A1 is the Ca-binding regulatory protein calmodulin (Siegel et al. 1982), although the physiological relevance of the A1-calmodulin interaction has not been demonstrated. A1 though such studies imply that A1 exerts its toxic effects by interaction with cytoplasmic components, it seems unlikely that A1 is soluble and toxic at the neutral pH of the cytoplasm, or that substantial amounts of A1 can enter thne cytoplasm through the non-polar harrier of the plasma membrane. It is possible that A1 acts to promote an external lesion, perhaps by blocking some essential site on the plasma membrane of the cell. However, due to the lack C suitable radioisotopes, studies of A1 uptake are difficult, and the cuestion of whether A1 has to enter the cell cytoplasm to exert toxic effects remains unanswered.

Because of its economic iTmportance, increasing A1 tolerance in crop plants would apDear to be an attractive target for molecular geneticists. Wide variation in A1 tolerance occurs naturally in plants, and it may be possible to decrease A1 sensitivity by the addition of an appropriate resistance gene, as has been done for other metal toxicities. For example, Cd tolerance of plants has been obtained by overexpression of a mammalian metallothionein protein (Pan et al, 1994). The most well characterised A1 -resistance trait is found in wheat (Delhaize et al, 1993). However, to date it has not Deen possible to clone this gene. Genetic engineering to improve A1 -sensitive species is thus restricted both by the lack of a clear molecular target for A1 -toxicity, and by the lack of suitable candidates for A1 -tolerance genes.

### OBJECT OF INVENTION

It is an object of this invention to isolate a gene associated with aluminium resistance and to at least partially identify the amino acid semence of the gene.

### DISCLOSURE

The applicant chose to use the yeast Saccharomyces cerevisiae to study the physiology and genetics of A1 stress. However, it should be appreciated that the invention is not limited to the isolation of A1 resistance in S. cerevisiae. Yeast has basic physiological similarities with plants (Serrano 1985). Metal tolerance has been studied in Saccharomyces, and mutants which show extra sensitivity or tolerance to metal ions have been isolated (for example Mehra et al. 1991). In some cases, metal tolerance genes have been isolated using an overexoression strategy (Conklin et al. 1994, Gaxiola 6t al. 1992). However, genetic analysis requires an appropriate selection for tolerant or sensitive strains. The present invention uses a selection for A1 tolerance in yeast to isolate two novel yeast genes which mediate resistance to A1³⁺, and describes their identification as homologues of bacterial proteins which transport divalent cations such as Mg^{2+ across the plasma membrane. The invention provides a gene which confers A}1 resistance when overexpressed in yeast. Preferably the gene is isolated from yeast.

In particular the invention provides the genes designated ALR1 and ALR2 as shown in FIG. 5 of the accompanying diagrams. The invention also provides the amino acid sequences of those genes and the proteins produced from these sequences.

The invention also provides yeast vector strains comprising one or both of the genes ALR1 /ALR2.

The invention also provides trarsgenic plants and animals containing an isolated gene which confers tolerance to A1. This gene may be ALR1 or ALR2, or any gene with functional homology to either or both of these genes, whether isolated from yeast, plants or animals.

The invention also provides a method of overexpressing a Mg transport gene from yeast in plants or animals to obtain A1 -tolerance. Resistance to other metals may be obtained by this method, such as resistance to trivalent cations (e.g. Ga, In, Sc etc), or to divalent cations (such as Mn, etc).

The invention also provides a method of isolating Mg transporters comprising selecting from plasmids or similar vectors expressing plant or animal cDNAs in yeast for clones that confer a high tolerance to A1.

The invention also Drovides a method of isolating Mg transporters comprising selecting from plasmiids or similar vecors expressing plant or animal cDNAs in yeast for clones that comtlement yeast strains with knock out mutations in ALR1 and/or ALR2 and/or ARH1.

The invention also provides the use of the isolated Mg transporter genes in the treatment of plant or animal diseases which result from a Mg deficiency in the plant or animal such as, or example, by producing an accumulation of Mg in plants deficient in Mg or in plants consumed by animals deficient in Mg.

The Mg transporter gene may be mutated in addition to and possibly in combination with its overexpression whicn may achieve better resistance to A1 , or improved cation transDort nroderties.

The invention also provides a method of isolating A1 tolerance genes from animals or plants, particularly wheat and rice, by selecting for clones that confer A1 tolerance among a library of zlasmids or other suitable vectors expressing plant or animal cDNAs in yeast.

The invention also provides a method of selecting for A1 tolerance in yeast comprising lowering the media pH in which the yeast are grown and decreasing the magnesium concentration to induce a sensitivity to A1 . Also provided are yeast strains selected by this method, the genes isolated from the yeast strains, and their amino acid sequences.

### BRIEF DESCRIPTION OF DRAWINGS

The invention will now be described, by way of examnle only, with reference to the drawings in which:

FIG. 1 shows the restriction digests of A1-resistance plasmids;

FIG. 2a shows the restriction map of pCGAB and deleion constructs;

FIG. 2b shows the restriction map and constructs derived from pSHA20 and pSHA29;

FIG. 3 shows the assignment of Rl1 and ALR2 to yeast chromosomes by CHEF gel electrophoresis and Southern hybyriaisation;

FIG. 4a shows the putative oven reading frames in the 12.5 kb secuence;

FIG. 4b shows the restriction map of 12.5 kb chromosome VI sequence showing the extent of the pCGA8 insert;

FIG. 5 shows a UWGCG LINEUP comparison of the ALR1, ALR2 and ARH1 hypothetical yeast divalent cation transporter proteins with bacterial homologues of the E. coli CorA protein;

alr1—partial protein secuence o ALR1 gene from pSMA20;

alr2 —yeast ALR2 protein

orf —yeast chromosome XI hypothetical 109.7 ka protein (Grebank accession number P35724)

Cora —E. coli Cor A protein (accession number L11042);

scora —S. tiphimurim Cor A protein (accession number L11043)

lecora —Mycobacteritm leprae Cor A homologue (accession number U15180)

bscora —b 3aci1lus subtilis Cor A homologue (accession number A30338)

mgtrans —consensus seuence

arh1=yeast ARH1 protein (earlier termed ORF)

syncoral=Synechocystis sp. CorA homologue 1380 aa, Genbank accession 1006592

syncora2 =Syrechocystis sp. CorA homologue 2387 aa, Genbank accession 1001431

### DETAILED DESCRIPTION OF THE INVENTION

### MATERIALS AND METHODS

Yeast strains and general techniques

Yeast strains used in this study are listed in Table 1.

TABLE 1
YEAST STRAINS

| Strain | Glenotype | Source or Reference |
|---|---|---|
| SH2332 | a pho3-1 pho4::HIS3 | S. Harashima |
| | his3-532 leu2-3 leu2- | |
| | 112 ura3-1,2 trpl-289 | |
| | ade2 | |
| CG379 | a ade5 can1 leu2-3 leu2- | YGSC* |
| | 112 trpl-289ₐura-352 gal2 | |
| | (Kil-0) | |
| DBY747-al | a ade2 his3Δ1 leu2-3 leu2-112 | P. Bergquist |
| | ura3-52 trpl-289ₐcan1 | |
| | GAL⁺ CUP^{r} | |
| FY23 | | |
| FY833 | | Winston et al 1995 |

*Yeast Genetic Stock Centre (UC Berkeley)

Yeast transformations were Derformed by the method of Gietz et al. (1992), a modification of the method of Schiestl and Gietz (1989). Escherichia coli DH10B [F′ merΔ (mrr-hsdRMS-merBC) ø80dlacLΔM15 ΔlacX74 deoR recA1 endA1 araD139Δ (ara, leu) 7597 galU galK Δ-rpsL nupG] (BRL) was used for plasmid construction and propagation. Standard yeast genetic techniques were described by Rose et al. (1990) .

Media

Standard YPD and SC media were prepared as described previously (Rose et al. (1990). Modified low phosphate, low A1 and low magnesium medium (LPM medium) was used for the A1-selection. LPM medium is based on the formalation of Difco “Yeast nitrogen base w/o amino acids” (Guthrie et al. 1991). LPM medium contains 200 μM MgCl₂, 100 μM KH₂PO₄ and has a final pH of 3.5. KC1 was used to replace phosphate and bring the final K⁺concentration to 5 mM. The medium was gelled by addition of 1% agarose (Sigma type II medium EEO). Glucose, vitamins and aluminum (as A1₂(SO₀₄)3) were added after autoclaving. For aluminium selections, aluminium was added to aive final concentrations of 100-250 μM A1³⁺.

Clonina and Sequencing Techniques

Yeast plasmid rescue was carried out by the glass bead method of Hoffman and Winston (1987). Cloning technicues were as described by Maniatis et al. (1982). DNA sequence analysis was performed using an ABI 373 automated DNA sequencer using dye-labelled terminators with double-stranded plasmid templates.

Hybridisations

Nucleic acid hybridisations were carried out by the method of Southern as described in Maniatis et al. (1982). Yeast chromosomes were orepared and separated using OFAGE gel apparatus according to standard methods (Rose et al. 1990). Probe DNA fragments were separated by agarose gel electrophoresis and purified using the Prep-a-gene kit (Bio-rad). DNA was labeled using [2−32P]dCTP, using a random primer labelling kit (BRL).

Plasmid Constructions

1) pCGA8 deletions and constructs

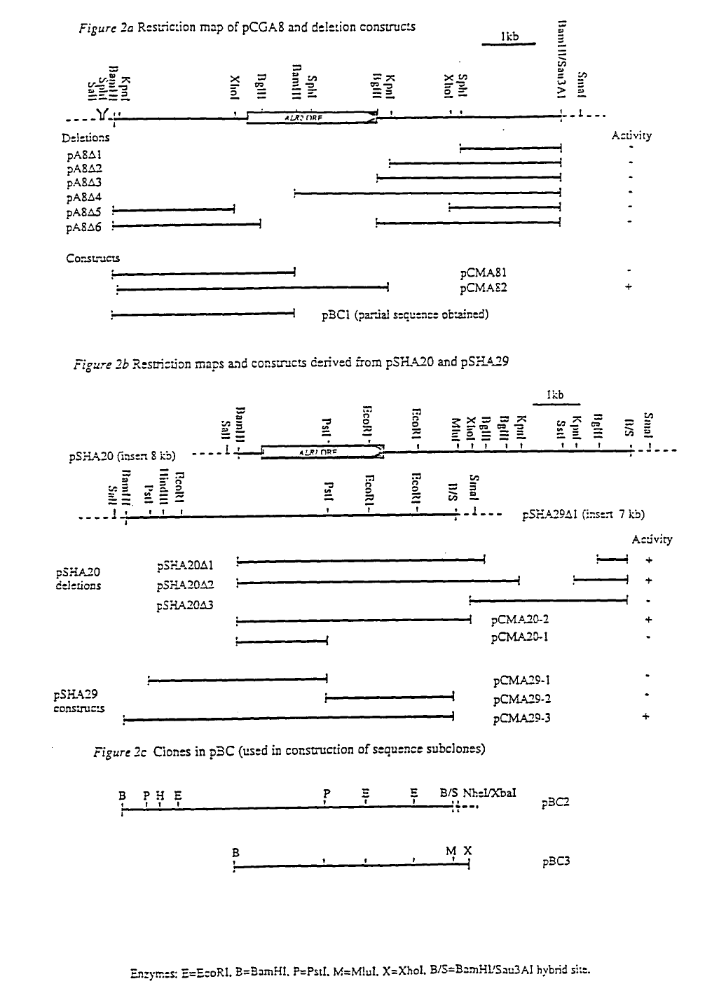

The yeast shuttle vectors pA8Δ1-Δ6 were constructed by digestion of pCGA8 at the enzyme sites shown in FIG. 2a, followed by religation of the vector to give the deleted derivative. For example, pA841 was constructed by digestion of pCGA8 with SphI to excise two insert fragments of 3 and 4 kb from the vector, which was then religated. Single enzymes were used in the construction of the deletions except in the case of pA8Δ3, which was digested with BamH1 and BglII. pCMA81 was constructed by digestion of pCGA8 with BamHI, gel isolation of the excised 3.8 kb ragment and ligation of the fragment into the shuttle vector pFI 46-S (Bonneaud et al. 1991) which had been digested with BaMHI. pCM82 was constructed by digestion of pCG-A8 with KpnI, gel isolation of the 5.2 kb fraament, and ligation into the KpnI-digested pFL44-S vector.

2) PSHA20 and pSHA29 deletions and constructs

The vectors pA20Δ1-3 were constructed by digestion of pSHA20 at the restriction sites shown in FIG. 2b, followed by religation of the vector. For example, pSHA20Δ1 was constructed by digestion of pSHA20 with BglII to excise two fragments of 2.1 kh and 0.45 kb respectively, followed by religation. pCMA20-1 and 20-2 were constructed as follows: pBC3, which consists of the 4.8 kb NarI/XboI fragment of pSHA20 ligated into the ClaI and XboI sites of the Stratagene pBC vector, was digested with PstI to excise a 1.9 kb fragment, which was then cloned into the PstI site of pFL46-S (Bonneaud et al. 1991) to give pCMA20-1. pCMA20-2 was constructed by excising the entire insert of pSHA29 with BamHI and XhoI and ligation of the 4.3 kb fragment into BamHI/Sal/I digested pFL44-S (Bonneaaud - al. 1991) .

pSHA29Δ1 (FIG. 2b) was constructed by digestion of pBCA29 with BamHI to exercise a 2 kb fragment from the SalI end of the insert, followed by religation. pCMA29-1 to pCMA29-3 were constructed using the vector pBC2, which consists of the 7 kb BamHI/NheI insert of PSHA20Δ1 subcloned into BamIII/XbaI digested pBC (Stratagene). This allowed the use of PstI and EcoRI sites in the insert without cleaving at these sites in YEp24. pCMA29-1 was constructed by digestion of pBC2 with PstI and ligation of the insert fragment into the PstI site of pFL46-S. pCMA29-2 is a construct consisting of the 2.2 kb PstI/SstI insert fragment of pBC3C2 cloned into PstI/SstI digested pFL46-S. pCMA29-3 contains the entire 7 kb insert of pBC3, excised with BamHI and SStI and cloned into BamIII/SstI-digested pFL46-S. The pYES/ALR1 plasmid was constructed by PCR amplification of the ALR1 open reading frame followed by cloning of the fragment into the pYES2 shuttle vector using the Xho1 and Not I sites included in the ALR1 oligonucleotide sequences

Construction of the ALR1, ALR2 and ARR1 deletion strains

The CM3 strain (alr2::URA3) was obtained using the one-step gene disruption method with the pCM3 plasmid. The insert of the pCM3 plasmid was excised by digestion with XhoI and used to transform strain FY833 to uracil prototrophy. Transformants were checked by Southern hybridisation with a probe to ALR2. The CM10 strain (alrl::HIS3) was obtained via the PCR disruption method. Two oligos were used to amplify a 1.1 kb product from the pHIS3 plasmid, which was used to transform a diploid strain to HIS3. Correct integration of the product was confirmed by Southern analysis using a probe specific to the ALR1 gene. in order to construct pCM/alr2::URA3, the XhoI fragment of the pCGA8 clone was excised and cloned into the pBC vector to constract pBC5. The central 2 kb BglII fragment of pBC5, which encompasses most of the reading frame of the ALR2 gene, was excised and replaced with a BglII fragment from the vector p-FL44, which contains the URA3 gene.

Results

Isolation of the ALRl and ALR2 aenes

The S. cerevisie strains SH2332 and CG379 (Table 1), differ in their basal A1-tolerance in LPM medium, and were used to select for plasmids which allow growth on inhibitory concentrations of aluminium. They were transformed with a yeast genomic library constructed in the high copy number shuttle plasmid YEₚ24 (Carlston and Botstein 1982). By selection for uracil prototropns, a total of 10,000 trans-ormants were obtained for each strain. The cells were resuspendend, washed twice with distilled water and approximately 50,000 transformants were plated on LPM (lacking uracil) medium with aluminium to screen for tolerant isolates. A1 was added to the plates to a level of 150 μM for SH2332 and 200 μM for CG379. Depending on the strain used, tolerant colonies were observed emerging from the background 3-6 days after plating. Initial A1 -tolerant isolates were restreaked to A1 -Dnlates to check their tolerance level, and the most tolerant clones were selected for further analysis.

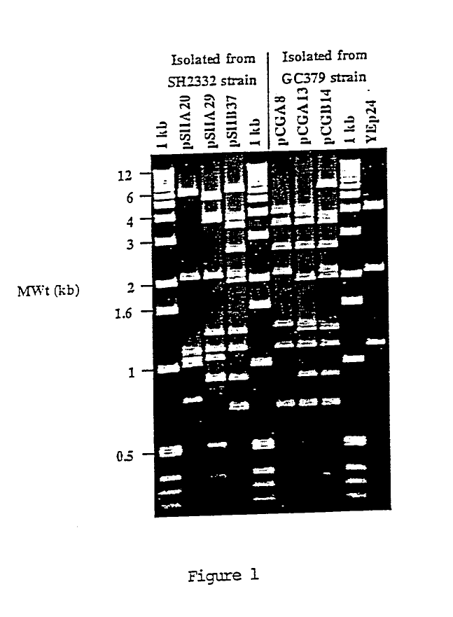

Crude Dreparations fo plasmid DNA were made from YPD cultures of several strains and the DNA used to transform the E. coli strain DH10B by electroporation. Plasmid DNA from the transformants was isolated and restriction mapping of the clones undertaken to characterise the inserts. Several clones with different restriction patterns were retransformed into SH2332 and CG379 to check they still conrerred A1 tolerance. Of the initial isolates, three plasmids (pCGA8, pSHA20 and pSHA29) which contained unique inserts as judged by restriction mapping and which conferred A1 tolerance upon retransformation, were selected for further analysis (FIG. 1). Plasmid DNTA was isolated from E. coli and g of DNA was digested with HindIII and EcoRI, then fractionated on a 1% agarose gel. 1 kb=1 kb DNA ladder (BRL). The gel shows the pSHA20, pSHA29 and pCGA8 plasmids as well as three other isolates with similar restriction matrs (pSHB37, pCGA13 and pCGB314). These isolates have restriction patterns indicating the presence of extra inserted DNA, probably resulting from recombination with the endogenous yeast 2 m plasmid.

Each of these these three plasmids functioned to increase A1 -tolerance in three different yeast strains (SH2332, CG379 and DBY747-al, Table 1), allowing growth on more than 250 M A1³⁺.

The three plasmids were further characterised by the use of Southern blotting and hybridisation. The results indicated that two of the plasmids (pSHA20 and pSHA29, FIG. 2b) contained inserts which overlapped (not shown). The third plasmid (pCGAS, FIG. 2a) appeared to contain a different DNA fragment as judged by hybridisation studies.

Localisation of the ALR1 and ALR2 genes by deletion construction

In order to delinea:e the location of the genes contained within the three clones, sections of the insert DN were removed from the plasmids, and the deleted clones tested for presence or absence or the gene activity (FIG. 2a, b). Six deletion clones were constructed from pCGA8 and transformed into CG379 (FIGS. 2a), and the transformants tested for presence or absence of growth on LPM+A1 plates. None of the six constructs conferred A1 tolerance. Two further constructs (pCMA81 and pCMA82) were made by subcloning fragments of the pCMA8 insert into yeast high copy shuttle vectors. pCMA81 did not confer A1 tolerance. The results suagested that the gene contained in pCGA8 was located in the central region of the yeast DNA fragment, and that the central BamHI site in the clone was located within the open reading frame of the gene.

When the restriction maps of the pSHA29 and pSHA20 clones were aligned, it was found that the overlapping region consisted of a region of yeast genomic DNA of approximately 4.5 kb (FIG. 2b). This overlap was confirmed by analysis of sequence information from subclones of the PstI and EcoRI fragments of pBC2 and pBC3. For example, sequencing of the 1.1 kb EcoRI fragment of pBC2 (derived from pSHA29) and pBC3 (derived from pSHA20) (FIG. 2c) subcloned in pBluescript indicated they were identical. This overlap suggested that the ALR1 gene resided within the 4.5 kb region. The gene localisation was confirmed by analysis of deletions and constructs of pSHA20 and A29 (FIG. 2b), and by subcloning the 4.5 kb fragment of pBC3, which contains much of the overlap region, into a yeast vector. This corstvact (pCMA20-2, FIG. 2b) was shown to confer A1 -resistance, and the resistance gene contained within was termed ALR1.

Mapping of ALR1 and ALR2 to yeast chromosomes by hybridisation

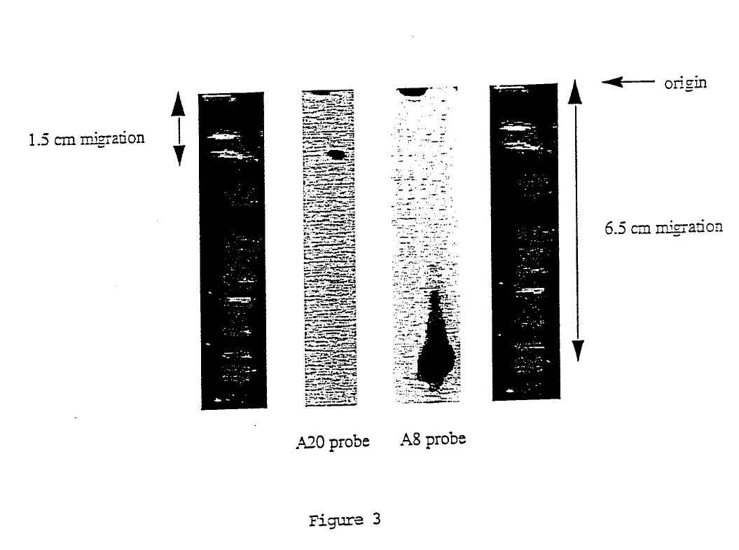

In order to map the two yeast genes to chromosomes, restriction fragments derived from the inserts of pBC1 (FIG. 2a and see below) and pBC3 were gel purified, labelled with 32p dCTP, and hybridised to a Southern blot of S. cerevisiae chromosomes (strain YPH45, Rose et al. 1990) which had been separated using the CHEF gel electrophoresis technique (Rose et al. 1990) (FIG. 3). The gel shown was blotted to a nylon membrane and hybridised to a 1.1 kb EcoRI fragment of pBC3 labelled with ³²p. The blot was developed with standard methods to give the first autoradiograph shown. After stripping, the process was repeated with a labelled 2.5 kb XhoI fragment of the pBCI plasmid to give the second autoradiograph. The 2.5 kb XhoI fragment of pBC1 hybridised to a band which had migrated 6.5 cm, and corresponded to chromosome VI. The 1.1 kb EcoRI insert of pBC3 hybridised to a band which had migrated 1.5 cm, corresponding to an unresolved doublet band of chromosomes VII and XV.

Identification of the probable ALR1 open reading frame

In order to obtain preliminary sequence information from the ALR1 gene, EcoRI and PstI restriction fraoent subclones were constructed from pBC2 and DBC3 (FIG. 2c) and short sequence taas obtained from the ends of the clones. These tags were used to search the public (EMBL. and Gebank) sequence databases in order to obtain information as to the possible function of the gene, and to check if the region containing the yeast gene had been sequenced as part of the international yeast genome sequencing project.

The open reading frame of ALR1 is nucleotides 416 -2995 in the DNA sequence found in Accession number u41293. The protein is 859 amino acids.

Localisation of the ALR2 gene by deletion construction and PCR

In order to delineate the location of the resistance genes within pCGA8, a series of six deleted clones were constructed (M&M). When tested in CG379, none conferred A1 tolerance. Two further constructs (pCMA81 and pCMA82) were made by subcloning fragments of the pCGA8 insert into high copy shuttle vectors. The pCMA2 construct conferred A1 tolerance, but pCMA81 did not. The results suggested that the gene contained in pCGA8 was located in the central region of the yeast DNA fragment, and that the central BamHI site in the clone was located within the open reading frame of the gene.

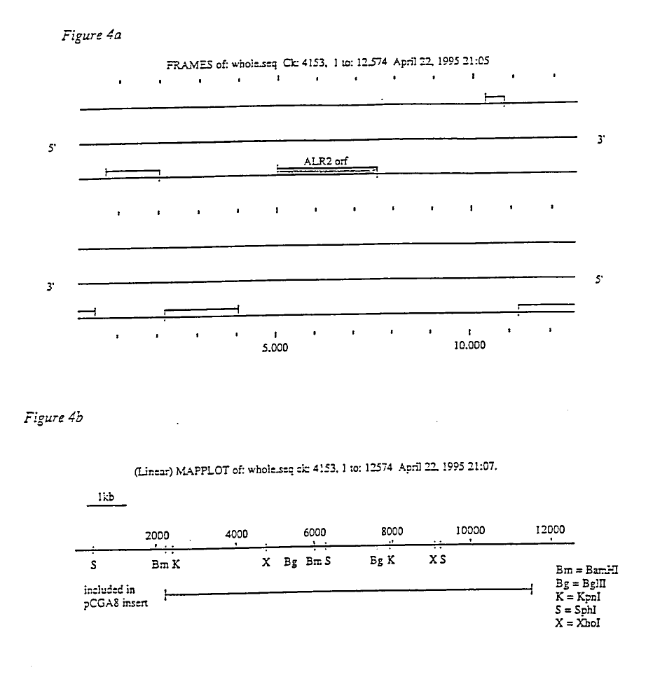

Identification of the ALR2 aene secuence The 3.8 kb BamHI fragment of pCGA8 was subcloned into the BamHI site of pBC (to give pBC1, FIG. 2a) and a partial sequence tag was obtained from each end. This was used to search both the public databases (using the BLASTX algorithm, Gish et al. 1993) and the confidential chromosome VI yeast seauence database at the Tsukuba Life Science Centre in Riken, Japan (pers. comm. Y. Murakami). Both sequence tags were found to be located in a secuenced region of chromosome VI, which contained a 2.6 kb unrknown open reading frame (FIG. 4a). This indicated that the pCGA8 clone contained a fragment of yeast chromosome VI, confirming the results of the chromosome mapping experiments. When the sequence of the 12 kb region surrounding the ORF was analysed using the UWGCG programme MAP to define restriction sites, it was found to have a similar restriction map as the pCGA8 insert (FIG. 4b), confirming the observed secuence homology and localising the clone to the left arm of chromosome VI.

Identification of the ALR2 gene and assignment of possible function

The 12.5 kb of sequence information obtained from Riken was analysed using the UWGCG program FRAMES, to find probable oven reading frames within the region covered by the insert of pCGA8. Of the three significant open reading frames which were found in the pCGA8 insert sequence, one could be identified as ALR2 on the basis of previous reletion analysis (FIG. 2a, 4b). The ALR2 gene has a reading frame of 2583 nucleotides, which encodes a protein of 860 amino acids. It has an accession number P43533, the DNA sequence is contained within accession number D44603 (gene ALR2 or YFLO5OC).

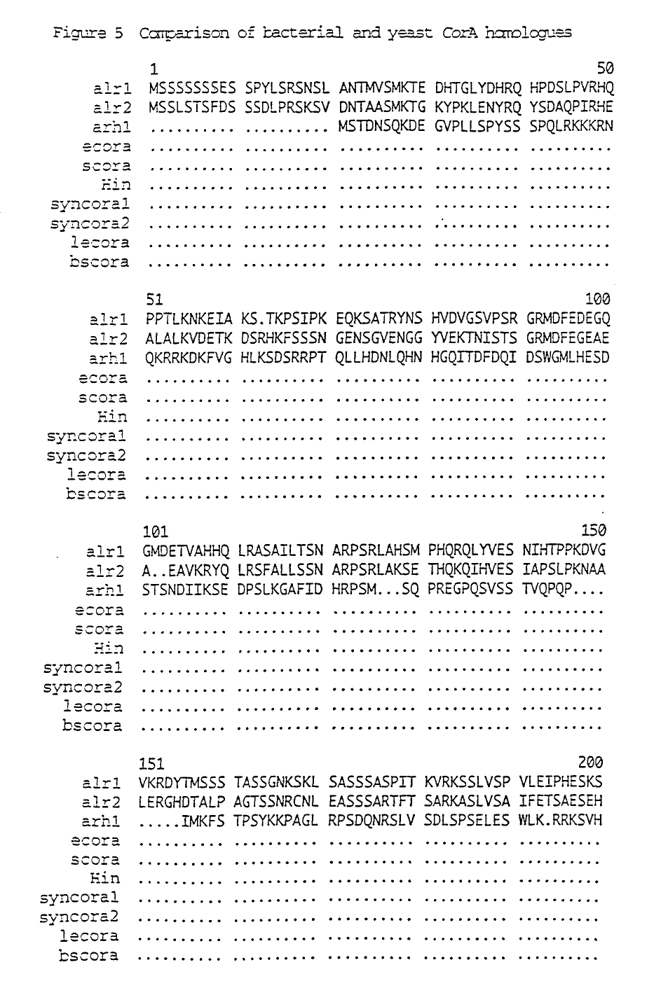

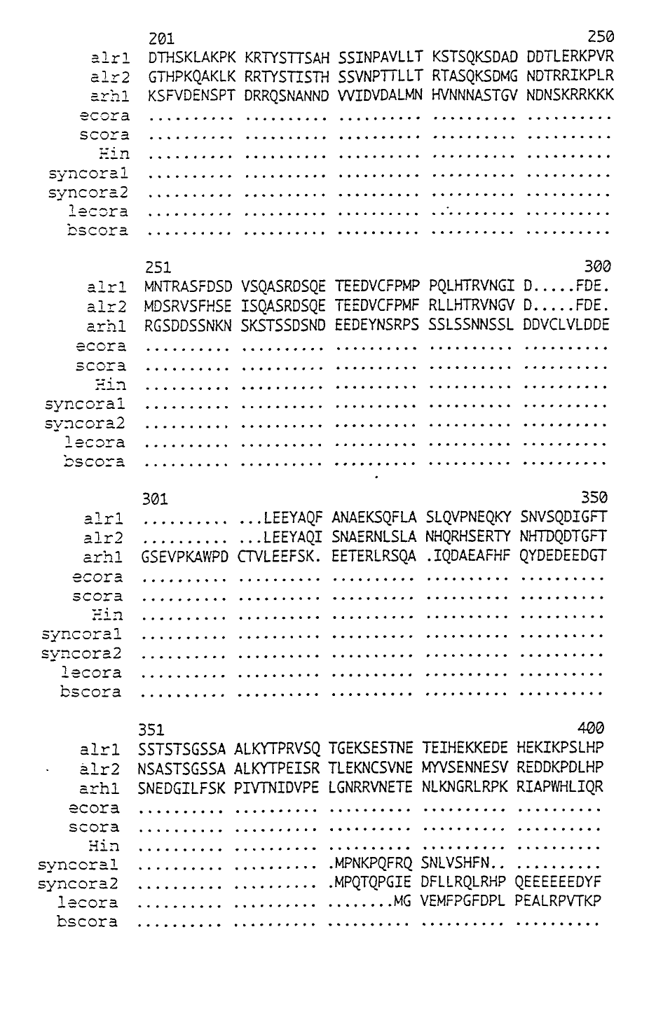

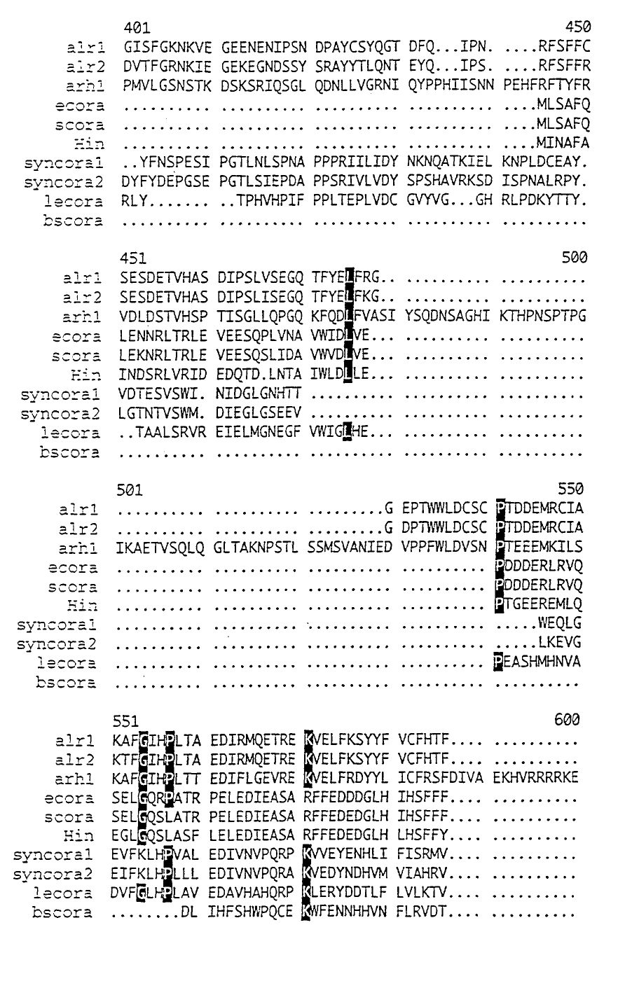

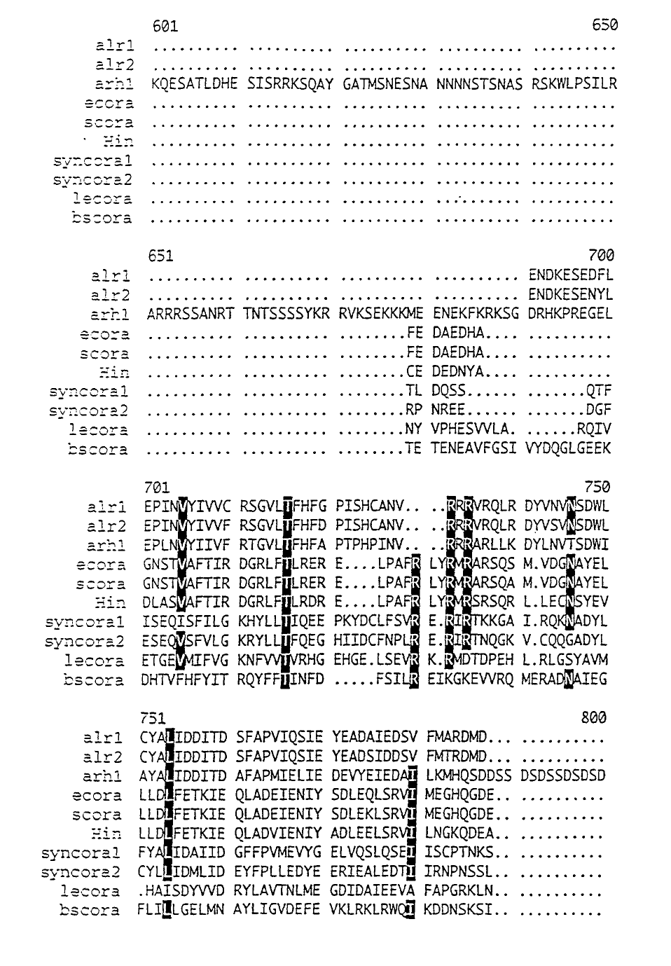

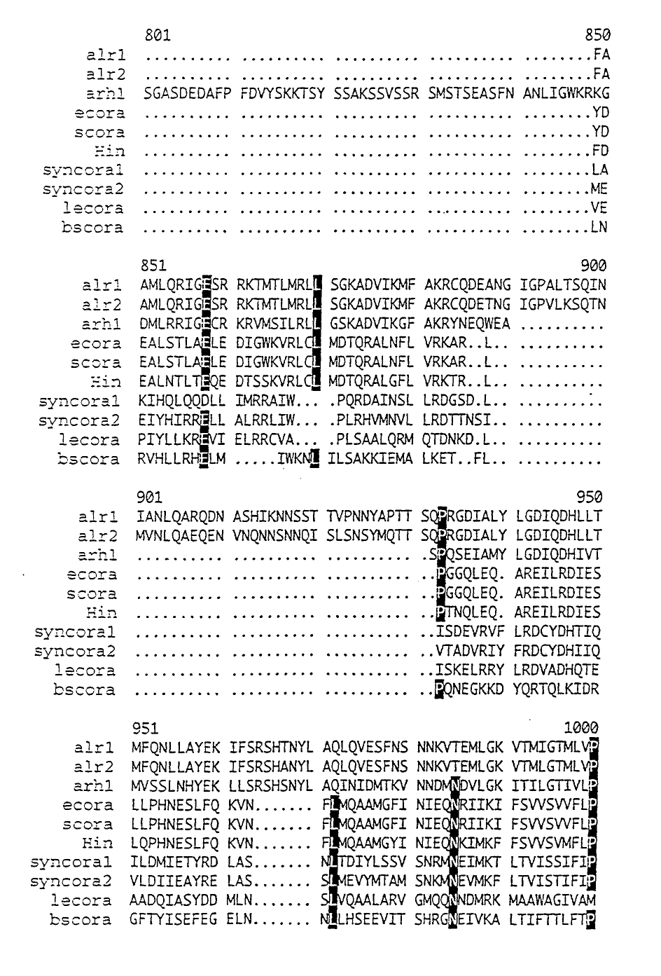

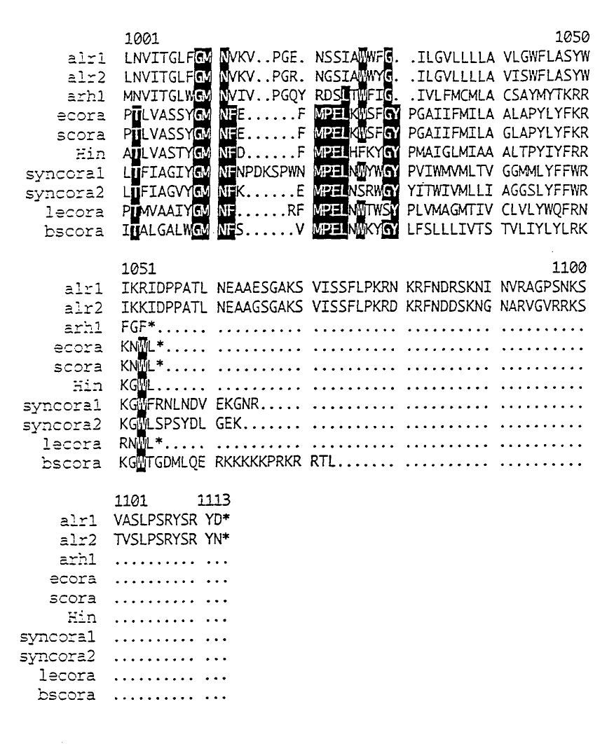

During examination of the ALR2 sequence it was found tat the ALR1 and 2 genes shared a high degree of homology, and both proteins were homologous to a 109.7 kDa yeast protein called ORF, or ARH1. The sequence is shown in FIG. 5. The three protein sequences could be easily aligned using the PILEUP algorithm (FIG. 5). Several areas of very strong conversation were found, particularly at the C-termini of the proteins. The length and sequence of the N-termini of the proteins was more variable, although all the proteins are all highly charged in this region.

The ALR2 peptide sequence was used to search the public sequence databases for similar proteins using the BLASTX program. The search revealed a low level of homology to the CorA gene from the bacterium Mycobacterium leprae. The M. leprae CorA gene was identified by its homology to the E. coli and Salmonella typhimurium CorA geres which have been show to encode proteins responsible for divalention uptake in these species (Smith et al. 1993). Several bacterial homologues of CorA have been suibmitted to Genbank, and these were obtained and compared with the yeast ALR2 and 109.7 kDa proteins, using the UWGCG program PILEUP (FIG. 5). A1 though the overall homology between the proteins was low, several areas of good conservation were identified, which were predominantly clustered at the C-termini of the proteins. These conserved regions correspond to the three membranes-spanning domains of the CorA protein previously identified and characterised by Smith et al. (1993).

A hydropathy plot of the ALR2 protein was Generated using the UWGCG program PEPLOT. The plot revealed three regions of the protein close to the C-terminus of the protein which could possibly participate in membrane-spanning domains (Klein et al. 1985). Comparison of hydrozathy plots of the ALR2 protein with the 109.7 kDa yeast protein and two bacteria CorA genes indicated all four proteins shared similar hydrophobic domains at their C-terimini, consistent with the secuence conservation observed in this region.

Dependence of ALR2 aene exression on strain background During deletion mapping of the ALR genes it was noted that the A1 tolerance of strains overexressing the two genes varied; pCGA8 —containing strains directly derived form S288C (such as the FY series) did not exhibit A1 tolerance, while miulticopy ALRI clones consistently conferred tolerance to all strains tested. In addition, both genes conferred tolerance on the CG379 and SH2332 strains. We suspected the ALR2 gene was not being exressed in the S288C background. To test our assessment, the ALR2 ORF was amplified from the pCGA8 plasmid and cloned into the exression cassette of the pYES2 vector, to give tne pYES/ALR2 vector. The resulting plasmid conferred high levels of A1 tolerance, regardless of the strain background. Although the pYES2 vector contains the GAL1 promoter, the plasmid still increased the A1 tolerance of strains growing on glucose, although tolerance was highest on galactose plates. The reason for incomplete catabolite regression of the GAL1p-ALR2 cassette in this plasmid is not known.

TABLE 2
GENE DISRUPTION AND COMPLEMENTATION
EXPERIMENTS SHOW ALR1 AND ALR2,
BUT NOT ARH1, TRANSPORT MAGNESIUM IONS

| | GROWTH ON SGaL* | GROWTH ON SGaL |
|---|---|---|
| STRAIN GENOTYPE | WITH 2 mM Mg | WITH 0.5 M Mg |
| alr1-Δ 1 | − | +++ |
| alr2-Δ 1 | +++ | +++ |
| arh1-Δ 2 | +++ | +++ |
| alr1-Δ 1, alr2-Δ 1 | − | +++ |
| alr1-Δ 1, arh1-Δ 2 | − | +++ |
| alr1-Δ 1 (pFL44-S) | − | +++ |
| alr1-Δ 1 (pFL38/ALR1) | +++ | +++ |
| alr1-Δ 1 (pFL38/ALR2) | − | +++ |
| alr1-Δ 1 (pFL44/ALR2) | + | +++ |
| alr1-Δ 1 (pYES/ALR2) | +++ | +++ |
| alr1-Δ 1 (pYES/ARH1) | − | +++ |

*SGaL = Synthetic galactose media without uracil

Materials and methods for Table 2 Plasmids used in these experiments were; pFL44-S (3Bonneaud et al. 1991). pFL38/ALR1, a low copy vector constructed by subcloning the entire insert of pBC3 (containing the ALR1 genomic clone) into the vector pFL38 (Bonneaud et al. 1991). pFL38/ALR2 constructed by subcloning the KpnI fragment of the pCGA8 plasmid containing the ALR2 genomic clone into the pFL38 plasmid (Bonneaud et al. 1991). pFL44/ALR2, a high copy vector constructed as for pFL38/ALR2, but using the pFL44-S vector (Bonneaud et al. 1991). pYES/ALR2 and pYES/ARH1, high level expression vectors constructed by PCR amplification and cloning of the ALR2 and ARH1 coding sequences into pYES2, as described in the legend to Table 3.

Strains were generated by standard genetic methods. The a1r1-Δ1 strains containing plasmids were isolated by transformation of the Mg-dependent alrl- Δ1 strain with plasmid DNA. The transformed strains were selected and propagated on media containing 500 mM MgCl₂ (liquid and solid YPDM, liquid and solid SCM-uracil). To test for Mg-dependency the strains were streaked to low and high Mg media (SGal-u) , and growth recorded after 4 days at 300C.

Gene Disruption of ALR1

The ALR1 gene was disrupted using the HIS3 gene with ALR1 homology introduced via PCR. Transformation of the havloid FY833 with the PCR construct resulted in non-specific integration of the fragment as judged by Southern analysis. Transformation of the diploid strain predominantly gave the correct single integration at the ALR1 locus. Sporulation of the CMl18 strain and tetrad dissection showed disruption of ALR1 was lethal on YPD medium (Table 2), since only his3 spores could be rescued. When the dizloid was transformed with a genomizc copy of the ALR1 gene on a URA3 plasmid and sporulated, HIS3/URA3 progency could be rescued, but not HIS3 alone.

ALR1 deletion results in Mg-dependent growth

In an attempt to rescue the lethality of the alr1-Δ1 allele, we dissected strain CM18 tetrads and incubated spores on media with high salt (100 mM CaCl₂-YPD), hypotonic conditions (1M sorbitol-YPD), low temperature (25° C.) and high MgCl₂ (100 mM, 500 mM and 1M MgCl₂-YPD). Rescue of HIS3 spore clones was found to he possible on 500 mM MgCl₂-YPD plates, although some growth was seen on 100 mM MgCl₂. None of the other conditions tested rescued the lethal phenotyp-e of the alr1-Δ1 allele.

Gene disruption of ALR2

The ALR2 gene was disrupted in an S288C background using the one step disruption method. The disruption plasmid pCM3 was constructed by insertion of the URA3 gene into the BalII sites of pCGA8. The hanloid strain FY833 was transformed with pCM3, which had been digested with XhoI and the 3 kb fragment isolated by gel purification. A high transformation frequency was obtained. When examined using Southern analysis, most of the transformrants were found to have anomalous patterns of transforming DNA fragments. This could be explained by the presence of two ARS sequences in the XhoI DNA clone used to disrupt the locus, which appear to allow independent replication of the pCM3 DNA introduced into the yeast cell. However, after screening multiple trarsformants, we obtained a strain which appeared to have the expected single copy insertion. The disrupted strain was viable, and did not display any increased sensitivity or tolerance to divalent or trivalent metals when examined by spot assays. In addition, the strain did not have any obvious nutritional requirements, and could mate and sporulate normally (not shown).

ALR2 can substitute for an ALR1 deletion

When the pYES/ALR2 plasmid was introduced into a alrl-Δ1 strain (CM22), the Mg-dependent phenotype was lost, and the strain grew normally on either glucose or galactose plates with 2 Mm Mg. It was also possible to partially alleviate the alr1-Δ1 phenotype by introduction of the genomic ALR2 multicopy construct, although ALR2 in single copy (pSL38/ALR2) was not effective (Table 2, pSL44/ALR2) or page 25.

Construction of the double mutant of ALR1 and ALR2

The CM22 (alr1-Δ1 ) and the CY3 (alr2-Δ1) mutant strains were mated on 500 mM Mg-CL-YPD plates and the diploid isolated and svorulated. As before, the HIS3 marKer segratad with dependence on 500 mM MgCl₂ for growth in both SD and YPD medium. The UR3 marker did not segregate with any noticeable phenotype. The double mutant strain CM23 could be isolated from the cross, and appeared to have a similar phenotype to the single alr1-Δ1 strain CM22. However, on closer examination, some slight differences in growth under various conditions were seen.

Amplification and cloning of the ARH1 open reading frame

The chromosome XI coding secuence homologous to ALR1 and ALR2 also resembled the bacterial CorA gene. For this reason we decided to examine the function of this gene and compare it to the other two CorA homologs in the yeast genome. The 3 kb ORF (YKL064W) (here identified as ARH1 for Aluminium Resistance Homolog 1) was amplified from yeast genomic DNA (strain FY833) and cloned into both pBC and the pYES2 expression vector (to give pYES/.ARH1). When FY833 was transformed with pYES/ARH1, the resulting strain was not tolerant to A1 or Ga in LPM medium. In addition, the pYES/ARH1 plasmid did not correct the Mg-dependency of an alr1- Δ1 strain. For this reason it appears that the ALR1 gene performs a different function to the ALR1 and ALR2 genes in yeast. ARH1 has a DNA secruence found within accession numoer D44605 (the gene is called YKL064W; the reading Frame is 109.7kDa).

Disruption of the ARH1 gene is not lethal

Using the coding sequence of ARH1 cloned in pBC we constructed a deletion derivative in which a central portion of the gene was replaced by the TRP1 marker. This construct was trans Iormed into the alr1-Δ1 divioid strain (CM18) and correctly disrupted transformants obtained. one strain was selected for further analysis. Upon sporulation of the disrupted diploid, a TRP1 haploid strain could be obtained. Using this strain, crosses were performed to construct tedouble mutant of ALR1 and ARH1. The double mutant strain could he rescued by dissection of triads to 0.5 M Mg/YPD plates, and appeared similar to the single alr- Δ1 mutant strain in its requirement for high Mg concentrations ,for growth.

Overexpressing ALR1, ALR2 and ARH1 effects the metal tolerance of yeast strains

We constructed a set of vectors designed to overexpress the ALR1, ALR2 and ARH1 genes from the regulated GAL1 promoter on plasmid nYES2. Strains containing these vectors were constructed by transformation and tested for growth on LPM plates containing different metal salts. The ,Petals tested included Co, Ni, Zn and Mm, divalent cations thought to be transported by the same system in yeast .(Fuhrmann and Rothstein); Cd and Cu, which are not thought to be transported by that system, and the itrivalent cations A1 , Ga, In, La and Sc. Where trivalent cations were used, strains were grown in LPM (100 μM Mg) to maximise toxicity of these metals. Dilutions of exponentially growing cultures were replicated to the various growth conditions, as described in materials and methods. Overexpression of all three genes gave rise to a range of metal sensitive and tolerant phenotypes, as shown in Table 3.

The two genes ALR1 and ALR2 both give resistance to A1 and to Ga, and make yeast cells sensitive to a range of other metals, including Zn, Co, Mn, Ni, La and Sc. The ARH1 gene confers a high degree of tolerance to Mn, but also gives sensitivity to Zn, Co, Ni Sc and La. It does not affect A1 or Ga tolerance. Two of the genes also slightly modify the growth response to Cd and Cu by mechanisms unknown.

TABLE 3
OVEREXPRESSION OF CorA HOMOLOGUES ALTERS
GROWTH RESPONSE UNDER TOXIC METAL STRESS
(a) Trivalent cations

| Gene | Al | Ga | In | La | Sc |
|---|---|---|---|---|---|
| ALR1 | ++ | + | NA | − − | − − |
| ALR2 | ++ | + | NA | − − | − − |
| ARH1 | NA | NA | NA | NA | NA |

(b) Divalent cations

| Gene | Co | Ni | Zn | Mn | Cu | Cd |
|---|---|---|---|---|---|---|
| ALR1 | − − | − − | − − | − − | − | + |
| ALR2 | − − | − − | − − | − − | − | + |
| ARH1 | − − | − − | − − | ++ | NA | NA |

NA = not affected
+, ++ increased tolerance compared to control strain
−, − − increased sensitivity compared to control strain

Construction of ALR1, ALR2 and ARH1 overexvression constructs in pYES2, and growth assay conditions.

The three galactose-reglated overexpression plasLids used were based on the pYES2 shuttle vector (Invitrogen). pYES2 is a high copy replicon in yeast (2 μm replication origin) in which cloned seqruences are expressed from the strong promoter of the yeast GAL1 gene. The pYES/ALR1 plasmid was constructed by PCR amplification of the ALR1 open reading frame using the High Fidelity PCR kit (Boehringer Marheim) with the pSHA20 plasmid as template, to give a product of 2646 nucleotides. Following restriction digestion of the XhoI and NotI sites in the oligonucleotide seouences (see table below), the product was cloned directly into the SalI and NotI sites oc pYES2. Both the ALR2 and 1 coding seauences were amplified using PCR with specific oligonucleotides (as described above) to give PCR products of 2634 and 3088 nucleotides respectively. These were digested at restriction sites in the oligonucleotides, cloned into the pBC vector (Stratagene), and checked by sequencing. The inserts were then sbdcloned into the SalI and NotI sites or pYES2, as described for ALR1, to give the pYES/ALR2 and pYES/AHR1 plasmids respectively.

The sequences of the oligonucleotides used in the PCP are listed below;

| Oligonucleotide Sequence (5′-3′) | | Used for |
|---|---|---|
| Alr1/3 | GGCCTCGAGCGAATATTGCTAGAAAGCGT | ALR1 |
| | | amplification |
| Alr1/4 | CGGCGGCCGCCACATCACTAATCAGTCGT | ALR1 |
| | | amplification |
| Alr2/1 | GGCCTCGAGCTTCGTAATGTCGTCCTTATC | ALR2 |
| | | amplification |
| Alr2/2 | CGGCGGCCGCAGATCTGCCGACCTACCATA | ALR2 |
| | | amplification |
| Arh1/1 | TAGCACTCGAGTCCCCTTACTTCGACAGTAA | ARH1 |
| | | amplification |
| Arh1/2 | TCAGATCTAGATCTATCCTTCGGGAAAGACA | ARH1 |
| | | amplification |

A yeast strain derived from s288c (FY834, Winston et al, 1995) was transformed with the three pYES2 constructs described above and a control plasmid (pFL44-S, 3Bonneaud et al. 1991). For growth tests, the four strains were growm to saturation in SC-uracil medium with glucose (Sherman 1991), then the cultures serially diluted 5-fold in distilled water and frogged to synthetic media plates containing galactose (2%) and metal salts. LPM medium (100 μM Mg, MacDiarmid and Gardner 1996) was used for plates containing trivalent cations, while divalent cations were added to low pH/low phosphate medium with 2 mM Mg (LPP plates). Strains were grown for 4-5 days at 30° C., then growth scored by comparison ine control strain (FY834/pFL-44-S).

Metals concentrations used were; Trivalents: A1 50 and 100 μM, Ga 100 μM, In 25 μM, La 500 μM, Sc5 μM.

Divalents: Co 1 μM and 2 μM, Zn 5 μM and 10 μM, Ni 250 and 500 μM, Mn 10 mM and 20 mM, Cl 100μM, Cd 10 μM and 20μM.

We believe that ALR1 and ALR2 transport into the cell Mg, Ni, Co, Zn, Mn, Sc and La, and that A1 and Ga inhibit this transoort. We believe that ARHl transports Ni Co and Zn into the cell, but may export Mn.

Mg transport

It is to be understood that the score of the invention is not limited to the described embodiments and therefore that numerous variations and modifications may be made to these embodiments without departing from the scope of the invention as set out in this specification.

INDUSTRIAL APPLICABILITY

Aluminium toxicity in plants, microorganisms and animals is a proolem. The isolation of two aluminium resistance genes will therefore find wide appliicability in conferring such plIants, microorganisms and animals aluminium tolerant. For examvle the use of the genes to poroduce transgenic, aluminium tolerant plants, microorganism and animals is envisaged. Wheat and rice transgenics are particularly envisaged. Aluminium tolerance genes could be isolated From yeast, plants or animals by overexpression in yeast using cDNA libraries in yeast overexoression vectors. Resistance to other trivalent cations is also possible. Due to the Mg-dependent growth phenotyoe of the strains disrupted in the ALR genes, a method of isolating such cation transporter genes is provided by complementation. The isolated cation transporter genes will find use in the treatment of animal and plant diseases resulting from cation deficiency.

The Mg transporter genes could be used to alter transport of Mg, Co, Mn, Zn, etc, in such a way as to overcome or modify symptoms of deficiency or toxicity of any of these elements in slants or animals, or to obtain high levels of these nutrients (accumulation).

For example isolation of a Mg transporter may be useful in the treatment of mid-crown yellowing of vine trees which is a result of Mg deficiency.

Alternatively Mg transporter genes could be used to treat Mg deficiencies in cows by the accumulation of Mg in cow's food such as ryegrass and clover. For example Mg transporter genes could be used in the construction of transgenic plants such as clover and ryegrass.

Alva KA, Asher CJ, Edwards DG (1986) The role of calcium in alleviating aluminium toxicity. Aust J Agric Res 37:375-382.

3aker JP, Schofield CL (1982) Aluminium toxicity to fish in acidic waters. Wat Air Soil Pollut 18: 289-309.

3Bonneaud N, Ozier-Kalogeropoulos O, Li G, Labouesse M, Mivielle-Sebastia L, Lacroute F (1991) A family of low and high copy replicative, integrative and single-standed S. cerevisiae/ E. coli shuttle vectors. Yeast 7:6)09 -615D ( 1991) .

Carlson M, Botstein D (1982) Two differentially related mNAs with different 5 ends encode secreted and intracellular forms of yeast invertasc. Cell 28: 145-154.

Conklin DS, Culbertson MR, Kung C (1994) Interactions between gene products involved in divalent cation transport in Saccharorryces cerevisa. Mol Gen Genet 244: 303-311.

Conner AJ, Meredith CP (1985) Simulating the mineral environment of aluminum toxic soils in plant cell culture. J Exot Bot 36: 870-880.

Dahigren Rk (1994) Soil acidification and nitrogen saturation From weathering of ammonium-bearing rock. Nature 368: 838-841.

Date R, Halliday J (1979) Selecting Rizobium for acid, infertile soils of the tropics. Nature 277: 62-64.

Delhaize E, Ryan PR, Randall PJ (1993) Aluminium tolerance in wheat (Triticum aestivum L.)II. Aluminium-stimulated excretion of malic acid from root apices. Plant Physiol 103: 695-702.

Foy CD (1988) Plant adaptation to acid, aluminium-toxic soils. Commun Soil Sci Plant Anal 19: 959-987.

Fuhrmann G F and Rothstein A (1968) The Transport of Zn²+, C02+and Ni²+into yeast cells. 3iochem, Biophys Acta 163: 325-330.

Qassmann W, Schroeder JJ (1994) Inward rectifying K+cnannels in root hairs of wheat. A mnechanism for aluminium-sersitive low affinity K+uptake. Plant Physiol 105: 1399-1408.

Gaxiola R, de Larrinoa IF, Villalba JM, Serrano R (1992) A novel and conserved salt-induced protein is an important determinant of salt tolerance in yeast. M3o J 1!: 3157-3164.

Haug A, Shi 3, Vitorello V (1994) A1 uminium interaction with phosphoinositide-associated signal trarsduction. Arch Toxicol 68: 1-7.

Hmiel SP, Snavely MD, Miller CG, Maguire y3 (1986) Magnesium transport in Salmonella typlimurium: characterisation of magnesium influx and cloning of a transport gene. Journal of Bacteriology 168: 1444-1450.

Hoffman CS, Winston F (1987) A ten-minute preparation from yeast efficiently releases autonomous plasmids for transformation of Escherichia coli. Gene 57: 267-272

Jackson GE (1982) Studies on the chnlation of aluminium for biological application, part 1. Citric acid. S Arr J Chem 35: 89-92

Jackson GE, Cosgrove A (1982) Studies on tne chelation of aluminium for biological application, part 2. Oxalic, mralonic and succinic acids. S Afr J Chem 35: 93-95.

Kinraide TB, Parker DR (1987) Cation amelioration of aluminium toxicity in wheat. Plant Physiol 83: 546-551.

Klein P, Kanchisa M, DcLisi C (1985) The detection and classification of membrane proteins. Biochim. Bionhys. Acta 815: 468-476.

Kochian KV (1995) Cellular mechanisms of aluminium toxicity and resistance in plants. Aknn Rev Plant Physiol Mol Biol 46: 237-260.

Luttge U. Clarkson DT (1992) Mineral nutrition: aluminium. In Progress in Botany, Vol. 53: 63-77. Springer Verlag, Berlin.

MacDonald TL, Humpreys WG, Martin LB (1987) Promotion of tubulin assembly by aluminium ion i, vitro. Science 236: 183-186.

Mariatis T. Fr,ritsch EF, Sambrook J (1982) Molecular cloning: a laboratory manual. Cold Spring Harbour Laboratory Press, Cold Spring Harbour, NY.

Mehra RK, Winge DR (1991) Metal ion resistance in fungi: molecular mechanisms and their regulated exression. J Cell Biochem 45: 30-40.

Pettersson A, Bergman 3 (1989) Effects of aluminium on ATP pools and utilisation in the cyanobacterium Anabera cylindrica: a model for the in vivo toxicity. Physiologia Plantarum 76: 527-534.

Pan A, Yang M, Tie F, Ti L, Chen Z and Ru 3 (1994), +7=ression oz mouse metallothionein-I gene confers cadmium resistance in transgeric tobacco plants. Plant Mol 3iol 24: 341-351.

Rengel Z (1988) Competitive inhibition of net Mg2+uptake by intact Lolium mul tiflcrum roots II. Plant ace effects. Plant Physiol 91: 1261-1267.

Rengel Z, elliott DC (1992) Mechanism of aluminium inhibition of net ⁴⁵Ca²+uptake by Amaranthus porotoplasts. Plant Physiol 98: 632-638.

Rengel Z, Robinson DL (1988) Competitive inhibition of net Mg²+uptake by intact Lolium multifloru m roots I. Kinetics. Plant Physiol 91: 1407-1413.

-Rose MD, Winston F and Heiter P (1990) Methods in Yeast Genetics: A Laboratory Course Manual. Cold Spring Harbour Laboratory Press. Cold Spring Harbour, New York.

Siegel N, Suhayda C, Haug A (1982) A1 uminium changes the conformation of calmodulin. Physiol Chem Phys 14: 165-167.

Sigler K, Knotkov A, Paca J, Wurst M (1980) Extrusion of metabolites from baker's yeast during glucose-induced acidification. Folia Microbiol 25: 311-317.

Smith Rl, Banks JL, Snavely MD, Maguire M- (1993) Secuence and topology of the CorA magnesium tansdort systems of Salmonella typhimurium and Eschericia coli. J. Biol.Chem. 26B: 14071-14080.

Snowden KC, Gardner RC (1993) Five genes induced by aluminium in wheat (Triticum aestivum L.) roots. Plant Physiol 103:696-701.

Suhayda CG, Xaug A (1986) Organic acids reduce toxicity in maize root mermranes. Physiol Plantarum 68: 189-195.

Wagerach W, O'Rourke K, Vitez L, Wieczorck A, Hoffmman S, Durfe S, Tedesco J, Stetler G (1991) Synthesis of wild type and mutant human haemoglobins in Saccharomyces cerevisiae. Biotechnology 9: 57-61

Winston F, Dollard C, Ricupero-Hovasse SL (1995) Construction of a set of convenient Sacchazromyces cerevisiae strains that are isogenic to S288C. Yeast II: 53-55.

1. A gene which confers aluminium resistance when overexpressed in yeast.

2. A gene as claimed in claim 1 which is isolated from yeast.

3. The gene ALRl which codes for the protein ALR1 as shown in FIG. 5 of the accompanying drawings, or functional equivalent thereof.

4. The gene ALR2 which codes for the protein ALR2 as shown in FIG. 5 of the accompanying drawings, or functional equivalent thereof.

5. The amino acid sequence corresponding to a gene as claimed in any one of claims 1-4.

6. The protein or peptide produced by the amino acid sequence as claimed in claim 5.

7. A yeast vector strain including a gene as claimed in any one of claims 1-4.

8. A transgenic plant, animal or bacteria containing an isolated gene which confers tolerance to aluminium ions.

9. A transgenic plant, animal or bacteria as claimed in claim 8 in which the isolated gene is a gene as claimed in any one of claims 1-4.

10. A transgenic plant, animal or bacteria as claimed in claim 8 in which the isolated gene is a gene with functional homology to a gene as claimed in any one of claims 1-4.

11. A method of isolating a cation transport gene comprising selecting from suitable vectors expressing plant or animal cDNAs in yeast for clones that confer a high tolerance to aluminium.

12. A method according to claim 11 in which the cation transport gene is a magnesium transport gene.

13. A method according to claim 11 or claim 12 in which selection is made for clones which complement ALR1 as shown in FIG. 5 of the accompanying drawings, or functional equivalent thereof.

14. A method according to claim 11 or claim 12 in which selection is made for clones which complement ALR2 as shown in FIG. 5 of the accompanying drawings, or functional equivalent thereof.

15. A method of isolating a cation transport gene comprising selecting from a vector expressing plant or animal cDNAs in yeast for clones that complement yeast strains with knock out mutations in ALR1 and/or ALR2 and/or AHR1 as shown in FIG. 5 of the accompanying drawings, or functional equivalents thereof.

16. A method according to claim 15 in which the cation transport gene is a magnesium transport gene.

17. The use of an isolated transport gene as claimed in any one of claims 11-16 in the treatment of any plant, animal or microorganism disease which results from a cation deficiency in the plant, animal or microorganism.

18. The use of an isolated transport gene as claimed in claim 17 which includes producing an accumulation of cations in plants deficient in those cations or in plants consumed by animals deficient in those cations.

19. The use as claimed in claim 18 in which the cations are magnesium cations.

20. The use of an isolated transport gene as claimed in any one of claims 11-16 in the treatment of any plant, animal or microorganism disease which results from cation toxicity in the plant, animal or microorganism.

21. The use as claimed in claim 20 in which the cation toxicity is manganese toxicity.

22. A method of overexpressing a cation transport gene in plants or animals to select aluminium tolerant plants or animals.

23. A method according to claim 22 in which the cation transport gene is from yeast.

24. A method according to claim 22 or 23 in which the cation is a magnesium cation.

25. A method according to any one of claims 22-24 in which the cation transDorter gene is mutated.

26. A method of isolating aluminium tolerance genes from microorganisms, animals, or plants comprising selecting for clones thrat confer aluminium tolerance from amongst a library of vectors expressing animal or olant cDNAs in yeast overexpression vectors.

27. A method of selecting for aluminium tolerance in yeast comprising lowering the pH and decreasing the magnesium concentration of the medium in which the yeast are arown to include a sensitivity of the yeast to aluminium and selecting those yeast strains which are aluminium tolerant.

28. A yeast strain selected by the method as claimed in claim 27 which is aluminium tolerant.

29. An aluminium tolerance gene derived from a yeast strain as claimed in claim 28, or functional equivalent thereof.

30. The amino acid sequence corresponding to the gene as claimed in claim 29, or functional equivalent thereof.

31. A transgenic plant, animal or bacteria containing an isolated gene which confers tolerance to manganese ions.

32. A transgenic plant as claimed in claim 31 in which the isolated gene is orf as shown in FIG. 5 of the accompanying drawings.

33. The use of an isolated transport gene as claimed in any one of claims 11-16 in combination with a pharmaceutical composition in the treatment of a disease.

34. The use as claimed in claim 33 in which the transport gene is a magnesium transport gene and in which the disease is a heart disease.
